(19) United States
(12) Patent Application Publication
(10) Pub. No.: US 2004/0249285 A1
(43) Pub. Date: Dec. 9, 2004

(54) Micro ultrasonic transducer building on flexible base substrate

(76) Inventors: Tse-Min Deng, Hsinchu City (TW); Tien-Chu Fan, Taichung City (TW); Gwo-Shiang Lee, Hsin-Tien City (TW); Ming-Wei Chang, Tai-Ping City (TW); Chih-Min Lai, Kaoshiung City (TW); Chia-Lin Chiu, Taipei Hsien (TW)

Correspondence Address: Keith Kline, PRO-TECHTOR INTERNATIONAL SERVICES, 20775 Norada Court, Saratoga, CA, 95070-3018, US

(21) Appl. No.: 10/648,495

(22) Filed: Aug. 25, 2003

(30) Foreign Application Priority Data
Jun. 9, 2003 (TW) 92210492

(51) Int. Cl. A61B 8/14

(52) U.S. Cl. 600/459

(57) ABSTRACT

A flexible ultrasonic transducer, comprising a plurality of ultrasonic transducer elements. Each of the ultrasonic transducer elements in turn comprises a base, a membrane and a first and a second electrode. The base is made of soft material, having an upper side and a lower side, with a support set on the upper side. The membrane is able to perform vibrations, having an outer side and an inner side, which is laid on the support. The first electrode is placed on the base and the second electrode placed in the membrane, the first and second electrodes being connected with a voltage source. Manufacturing the flexible ultrasonic transducer requires less steps without increasing cost. By having a flexible shape, emission and reception of ultrasonic waves as well as an effective area are enhanced, with less attenuation of ultrasonic waves, resulting in more effective sensing.

### BACKGROUND OF THE INVENTION

1. Field of the Invention

The present invention relates to an ultrasonic transducer on a flexible substrate, particularly to a micro ultrasonic transducer on a flexible substrate with enhanced emission and reception of ultrasonic waves, which, by increasing an effective transmission area, improves coupling to air, reducing attenuation for high responsiveness to frequency changes, effective energy transformation and detecting speed, allowing various sound speeds, geometrical shapes and focal lengths for a wide range of applications.

2. Description of Related Art

Conventional ultrasonic transducers transform electrical energy into mechanical energy, having a sensor head emitting an elastic wave into a bulk body and receiving an elastic wave, which has been reflected at an internal surface.

Ultrasonic sensors are widely used central components of ultrasonic detectors. Ultrasonic detectors undergo the current trend of miniaturisation, which offers the advantages of high response, high resolution and a wide range of applications. A conventional ultrasonic detector depends for functioning on proper coupling. Mostly a coupling medium is required, like a liquid or a solid body. If air is used as a coupling medium, a layer glued on a back side and an adaptive layer have to be incorporated into the design. Current characteristics of air have an influence on the properties of the sensor. For transmission on curved surfaces, Snell's law has to be taken into account, which describes intensities of the original, the reflected and the transmitted waves depending on the incidence angle. On curved surfaces, effective inspection areas are thereby possibly diminished, and long paths of detected waves result in decreased signals and increased variable conditon of air coupling, as shown in FIG. 5. Furthermore, upon an inclined direction of incidence, as shown in FIGS. 6 and 7, a larger inclination angle θ₁ of the transmitted wave results in further reduced transmission, distorting measurement results, possibly to the point of no detection of signals. Therefore, for good resolution and stability, it is indispensable to develop a transducer which is effective at surfaces bordering air and overcomes the problem of insufficient transmission on curved surfaces.

Transmissibility of ultrasonic waves at the couplant of liquid and solid bodies is influenced by temperature, pressure, density and velocities of particles, limiting the range of applications. With changed air coupling medium, for the mismatch of transducer impedance, intensities of waves as well as frequency responses, band width and direction are reduced, hampering sensor functions. Furthermore, curved surfaces result in further reductions of transmission of waves.

Presently, micro ultrasonic devices limit of geometry, lead the wave effective transmission are manufactured using micro techniques which promise to reduce costs and allow for mass production. Therein, silicon is used as basic material, and a range of complex techniques is employed. Materials used are brittle and are not readily formed into required shapes, so that intensities and efficient do not reach anticipated values. U.S. Pat. No. 6,328,697B1 discloses an array of ultrasonic detectors 90 having several layers, as shown in FIG. 8. On a base 91 of silicon a surface is built at a low temperature. A support 92 of Si₃N₄ is grown thereon from PECVD process. After that, material that is not needed is removed, so that a membrane 93 on the support 92 remains. A lower electrode is formed by the conductivity of the silicon base 91, and an upper electrode 94 of aluminum is grown on the membrane 93 from vapor, forming a protective layer. A characteristic of this device, however, lies in using silicon as material of the base and using a hard material for the support. Therefore, there is no way to bend the ultrasonic detectors 90. While manufacturing can be performed in an integrated process and adaption is improved, attenuation is high and resolution is reduced, as compared to a device on a flexible base, so there is still a need for improving on the above mentioned problem.

### SUMMARY OF THE INVENTION

The object of the present invention is to provide a flexible ultrasonic transducer which is rapidly produced and offers low attenuation and match layers with little impairing of transmission. Therein, a flexible metallic base carries supports on two sides, on which a metallic membrane is laid performing vibrations. Employing the supports generates a relatively thick insulating layer, simplifying manufacturing. Between two metallic electrodes thus formed, a larger amount of energy is stored for driving the membrane. Attenuation is reduced and adaptability is improved, resulting in more effective detection.

The present invention can be more fully understood by reference to the following description and accompanying drawings.

### DETAILED DESCRIPTION OF THE PREFERRED EMBODIMENTS

As shown in FIGS. 1 and 2, the flexible ultrasonic transducer of the present invention comprises a base and a plurality of ultrasonic transducer elements 1, which in turn comprise: a membrane 20; a plurality of first electrodes 31; and a plurality of second electrodes 32. The base 10 has an upper side 11 and a lower side 12. For each ultrasonic transducer element 1, a depression 13 cuts into the upper side 11, with a support 14 set on two lateral sides thereof The upper and lower sides 11, 12 and the support 14 are made of flexible material or rigid material. For each ultrasonic transducer element 1, the base 10 and the support 14 form a single body made of uniform material, e.g., silicon, Si₃N₄, polysilicon, kapton, nickel, teflon, resin, plastics, polyester, photoresist or polymolecular material. The support 14 has an upper end with a top side 15. The membrane 20 has an outer side 21 and an inner side 22, which is placed on the top side 15 of the support 14. By combining a plurality of ultrasonic transducer elements 1 on an extended base 10 the flexible ultrasonic transducer of the present invention is formed. The plurality of first and second electrodes 31, 32 are made of gold, silver, nickel, aluminum and copper. At the location of each ultrasonic transducer element 1, one of the plurality of first electrodes 31 is inserted between the upper and lower sides 11, 12 of the base 10. If the base 10 is made of electricity conducting material, the first electrode 31 is not needed and dispensed with. In the same way, for each ultrasonic transducer element 1, one of the plurality of second electrodes 32 is inserted between the outer and inner sides 21, 22 of the membrane. The plurality of first electrodes 31 and the plurality of second electrodes 32 are respectively connected with a voltage source. The plurality of first electrodes 31 can also be installed on the upper side 11 or the lower side 12 of the base 10, and the plurality of second electrodes 32 can also be installed on the outer side 21 or the inner side 22 of the membrane 20.

In the arrangement just described, the support 14 has a relatively large height, consequently, the distance between the first and second electrodes 31, 32 is relatively large, providing a relatively thick insulating layer and allowing for a large vibration amplitude of the membrane, furthermore reducing the steps needed for manufacturing the silicon base. Between the first and second electrodes 31, 32, a relatively large amount of electrical energy is stored for driving the membrane 20 to be converted to mechanical energy for vibrations.

Referring to FIG. 2, various ultrasonic transducer elements are distributed with equal mutual distances. Using soft material for the base 10 and a large number of ultrasonic transducer elements results in a high amount of mechanical energy for vibration.

Referring to FIGS. 3 and 4, with the base 10 being flexible, high transmission of ultrasonic waves are attained. Coupling to air and transmitting energy, attenuation is compensated and a high response by any type of sensor is achieved.

The present invention discloses a flexible ultrasonic transducer, using conventional height-width-depth relations and microelectronic control (e.g., but not limited to DRIE-, LIGA-like technology), comprising a micro-array, flexible supports and transducers and being connectable with all types of external or integrated electric circuits.

As the above explanation shows, the flexible ultrasonic transducer of the present invention, without increasing manufacturing cost, allows faster production, enhanced vibration with a higher effective area, less attenuation and better adaption.

While the invention has been described with reference to preferred embodiments thereof, it is to be understood that modifications or variations may be easily made without departing from the spirit of this invention which is defined by the appended claims.

### BRIEF DESCRIPTION OF THE DRAWINGS

FIG. 1 is a schematic side view of one of the ultrasonic transducer elements of the present invention.

FIG. 2 is a perspective view of the flexible ultrasonic transducer of the present invention.

FIG. 3 is a schematic illustration of adaption and attenuation of ultrasound waves on a curved surface using the present invention.

FIG. 4 is a schematic illustration of angular transmission of ultrasound aves on surfaces using the present invention.

FIG. 5 is a schematic illustration of signal refections on a curved surface using a conventional ultrasonic transducer.

FIG. 6 is a schematic illustration of adaption and attenuation of ultrasound waves on a curved surface using a conventional ultrasonic transducer.

FIG. 7 is a schematic illustration of angular transmission of ultrasound waves on a curved surface using a conventional ultrasonic transducer.

FIG. 8 is a schematic side view of a conventional ultrasonic transducer element.

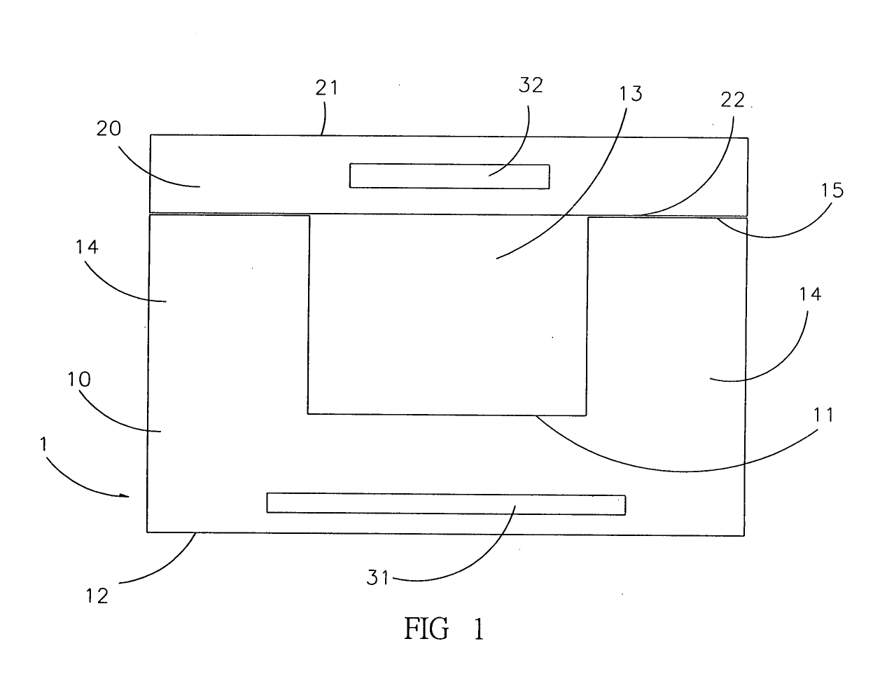

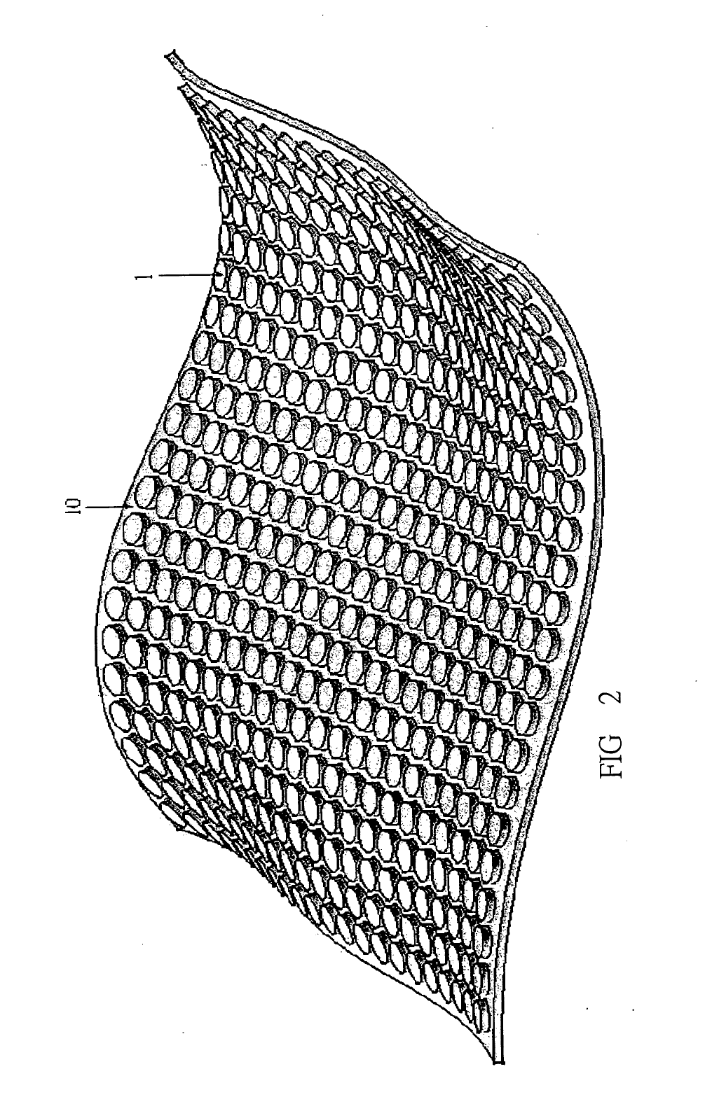

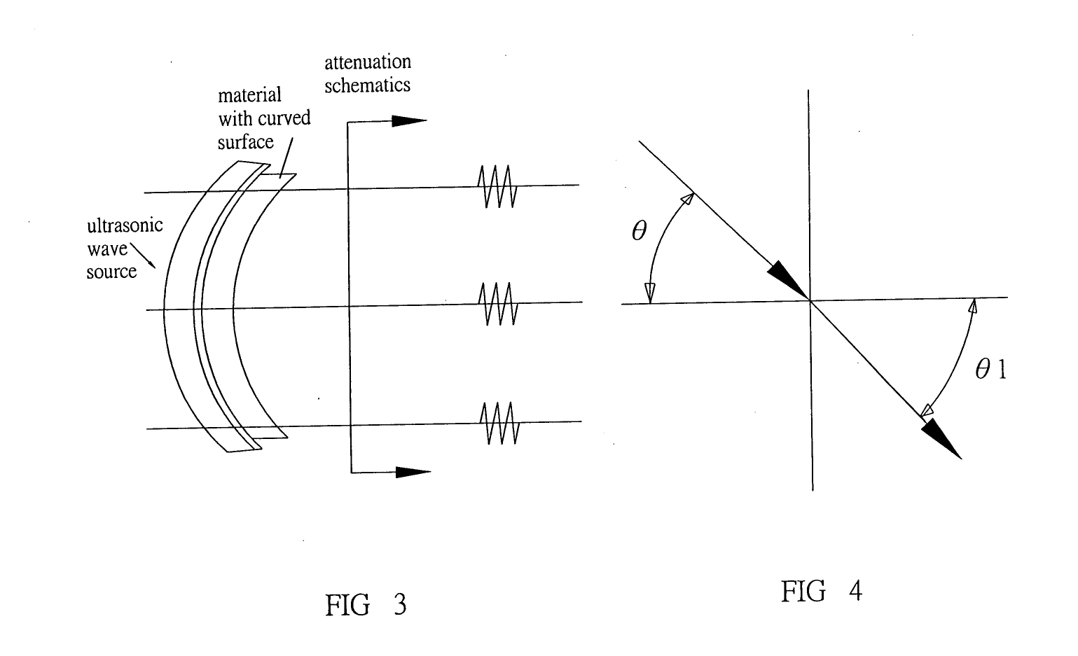

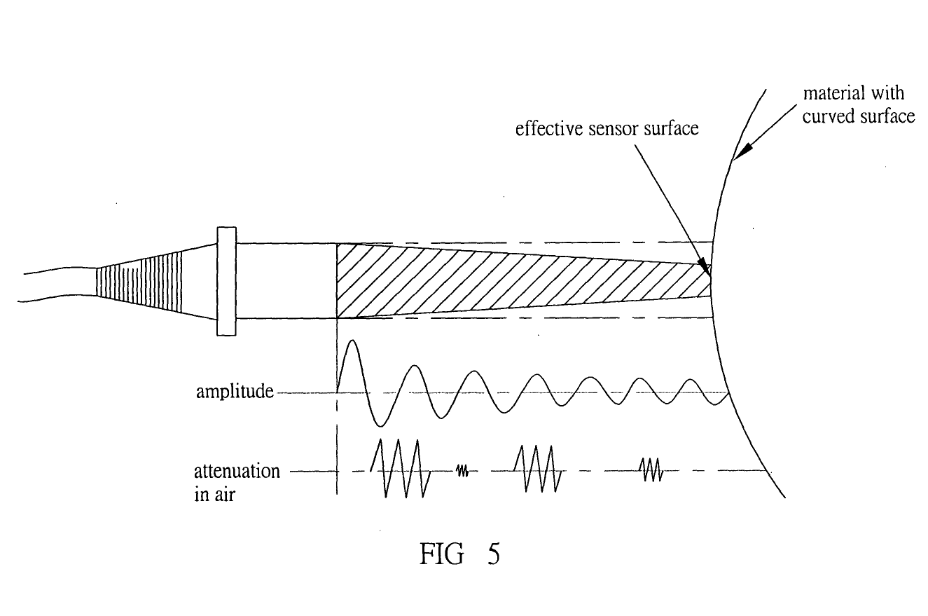

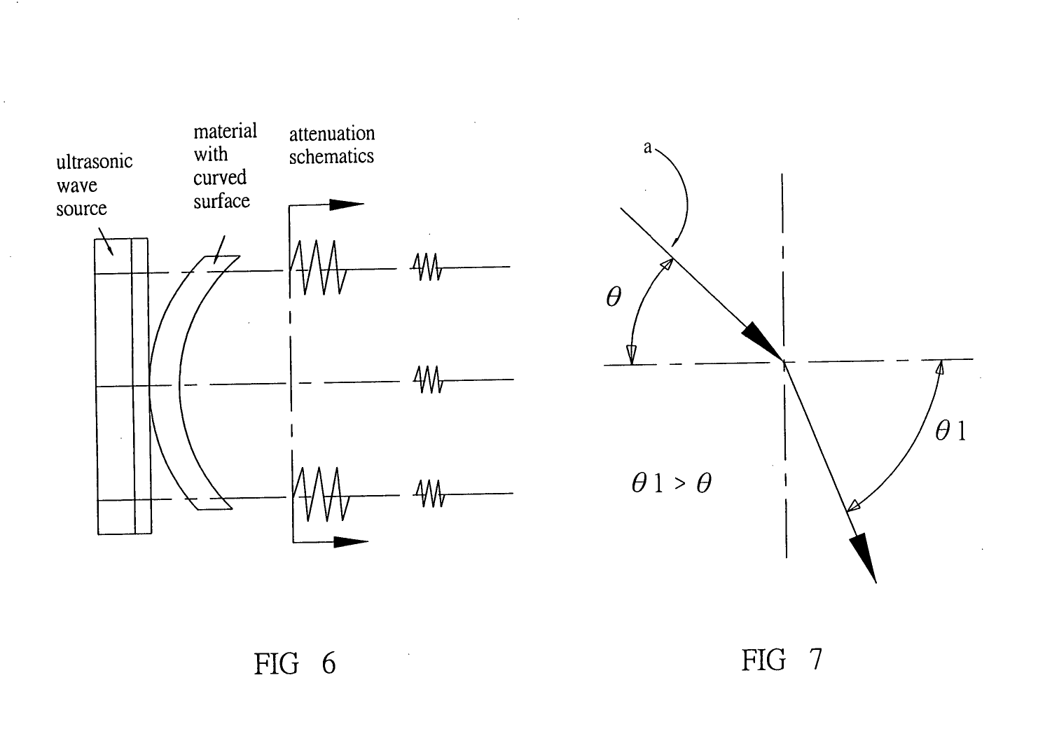

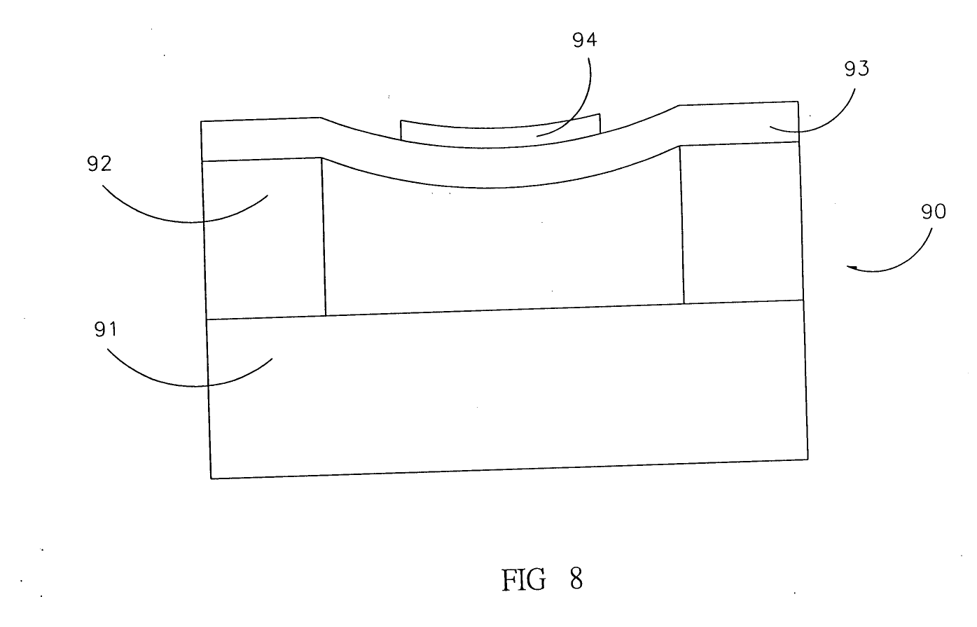

1. A flexible ultrasonic transducer, comprising a plurality of ultrasonic transducer elements, each of said ultrasonic transducer elements in turn comprising:
a base, made of flexible material, having an upper side and a lower side, with a support set on said upper side on two lateral sides thereof;
a membrane, able to perform vibrations, having an outer side and an inner side, which is laid on said support;
a first electrode placed on said base and a second electrode placed in said membrane, said first and second electrodes being connected with a voltage source;
wherein manufacturing steps are reduced without increasing cost and, by said substrate being flexible, emission and reception of ultrasonic waves as well as an effective area are enhanced, with less attenuation of ultrasonic waves, resulting in more effective sensing.

2. The flexible ultrasonic transducer according to claim 1, wherein said upper side of said base has a depression.

3. The flexible ultrasonic transducer according to claim 1, wherein said base is made of at least one nonuniform material.

4. The flexible ultrasonic transducer according to claim 1, wherein said membrane is made of at least one nonuniform material.

5. The flexible ultrasonic transducer according to claim 1, wherein said membrane is made of at least one material.

6. The flexible ultrasonic transducer according to claim 1, wherein said first electrode has an electricity conducting element inserted in said base.

7. The flexible ultrasonic transducer according to claim 1, wherein said first electrode is inserted between said upper and lower sides of said base.

8. The flexible ultrasonic transducer according to claim 1, wherein said second electrode is inserted between said outer and inner sides of said membrane.

9. The flexible ultrasonic transducer according to claim 1, wherein said support has a top surface.

10. The flexible ultrasonic transducer according to claim 9, wherein a membrane is placed on said top surface of said support.

11. The flexible ultrasonic transducer according to claim 10, wherein said inner surface of said membrane is placed on said top surface of said support;

12. The flexible ultrasonic transducer according to claim 1, wherein said support is made of flexible material.

13. The flexible ultrasonic transducer according to claim 1, wherein said base is made of electricity conducting material.

14. The flexible ultrasonic transducer according to claim 1, wherein said base, if made of electricity conducting material, houses said first electrode, said first and second electrodes being connected with a voltage source.

15. The flexible ultrasonic transducer according to claim 1, wherein said base and said support form an integrated body.

16. The flexible ultrasonic transducer according to claim 14, wherein said first and second electrodes are made of gold, silver, copper, nickel or aluminum.

17. The flexible ultrasonic transducer according to claim 1, wherein said base, said support and said membrane are made of silicon, Si₃N₄, polysilicon, kapton, nickel, teflon, resin, plastics, polyester, photoresist or polymolecular material.

18. The flexible ultrasonic transducer according to claim 1, wherein said support and said base are made of flexible material.

19. The flexible ultrasonic transducer according to claim 1, wherein said support and said base are made of different materials, one of which is not flexible.
